(19) United States
(12) Patent Application Publication
(10) Pub. No.: US 2003/0198609 A1
(43) Pub. Date: Oct. 23, 2003

(54) Use of ectoin or ectoin derivatives for the prophylaxis and/or treatment of uv-induced immunosuppression

(76) Inventor: Joachim Bünger, (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/239,073

(22) PCT Filed: Mar. 15, 2001
(86) PCT No.: PCT/EP01/02988

(30) Foreign Application Priority Data
Mar. 24, 2000 (DE) 100 14 631.7

(51) Int. Cl. A61K 38/05; A61K 38/04; A61K 31/505; A61K 7/42; A61K 31/4172

(52) U.S. Cl. 424/59; 514/18; 514/19; 514/256; 514/401

(57) ABSTRACT

The present invention relates to the use of at least one compound selected from the group comprising compounds of formulas 1a and 1b

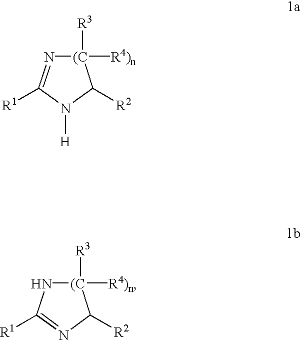

physiologically acceptable salts thereof and stereoisomeric forms thereof,

in which

R¹ denotes H oder alkyl,

R² denotes H, COOH, COO-alkyl or CO—NH—R⁵,

R³ and R⁴ each independently denote H or OH,

n is 1, 2 or 3,

R⁵ denotes H, alkyl, an amino acid group, a dipeptide residue or a tripeptide residue, and

alkyl denotes an alkyl group containing from 1 to 4 carbons,

for the prophylaxis and/or treatment of immunosuppression.

These compounds are used in the present invention in the form of a topical composition.

The present invention relates to the use of ectoin or ectoin derivatives for the prophylaxis and/or treatment of UV-induced immune suppression.

The skin, as the boundary surface of the human body, is exposed to a large number of environmental stress factors. The human skin is an organ which protects the body from external influences by means of a variety of specialized cell types, such as the keratinocytes, the melanocytes, Langerhans cells, Merkel's cells, and embedded sense cells. These external influences on the human skin should be distinguished according to whether they are of a physical, chemical, or biological nature. The physical external influences include thermal and mechanical effects and also the action of radiation, such as UV and IR radiation. By chemical external influences are meant, in particular, the action of toxins and allergens. Biological external influences comprise the action of foreign organisms and their metabolic products. Other stress factors include pathological states and diseases, such as pyrexia, inflammation, infection, cell and tissue trauma, and also physiological processes, such as cytokinesis.

The human skin possesses a specific immunological defence system, the so-called skin immune system. In and near the epidermis the Langerhans cells (LC) exercise a key function in the immune system of the skin, which serves the purpose of protecting the human organism from damaging environmental factors, pathogens and transformed skin cells.

When the human skin is exposed to the sun under severe conditions there is weakening of the skin immune system caused, in particular, by the action of the UV-B radiation and near-UV-B UV-A radiation present in sunlight. This phenomenon is known as UV-induced immunosuppression. (M. L. Kripke, Adv. Cancer Res. 34, 1981, 69-106). The epidermal LCs, being cellular key elements of the skin immune system, are particularly affected by this UV-induced immunosuppression. Approximately 2 to 4% of the epidermal cells are Langerhans cells, which are normally located in the suprabasal region of the epidermis and have a dentritic morphology. The branches of the LCs, known as dentrites, extend up to the top layers of the epidermis, such as the Stratum granulosum, and form a dense network permeating the entire epidermis. LCs can be identified not only by their morphology but also by the use of the histological markers HLA-DR, CD1a, CD4 and membrane-ATPase. Following exposure to sunlight or UV irradiation, the LCs in the human skin undergo the following impressive and significant changes:

The number of LCs drops in accordance with the UV dose applied. The LCs lose their dentritic morphology and become more rounded. Furthermore, the LCs lose their antigen-presenting properties.

Thus exposure to strong sunlight leads to immunosuppression in the skin.

It is thus an object of the present invention to overcome, or at least alleviate, the aforementioned problems and to provide a compound which is suitable for the prophylaxis and/or treatment of UV-induced immunosuppresion.

This object is achieved by the use of at least one compound selected from the group comprising compounds of formulas 1a and 1b:

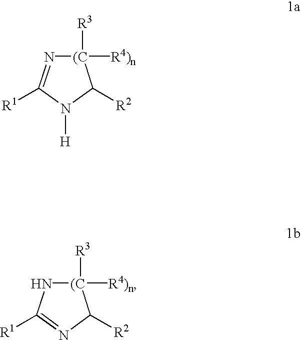

physioligically acceptable salts thereof, and stereoisomeric forms thereof,

in which

R¹ denotes H oder alkyl,

R² denotes H, COOH, COO-alkyl or CO—NH—R⁵,

R³ und R⁴ each independently denote H or OH,

n is 1, 2 or 3,

R⁵ denotes H, alkyl, an amino acid group, a dipeptide residue, or a tripeptide residue, and

alkyl denotes an alkyl group containing from 1 to 4 carbons,

for the prophylaxis and/or treatment of UV-induced immunosuppression.

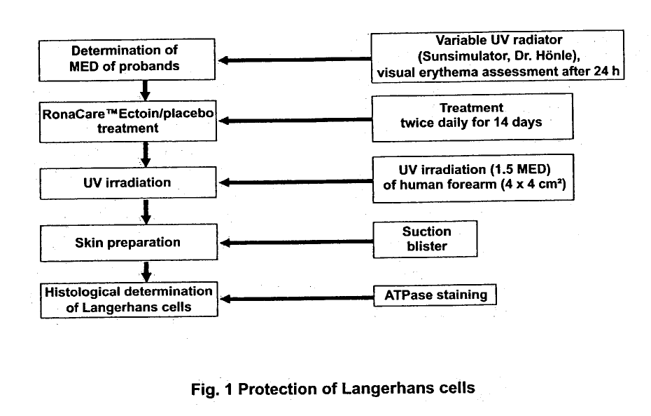

FIG. 1 is a diagrammatic overview of an experiment for proving the efficacy of ectoin as a protector of Langerhans cells, as carried out in Example 14 and Comparative Example 1.

The compounds of formulas 1a and 1b, the physiologically acceptable salts of the compounds of formulas 1a and 1b, and the stereoisomeric forms of the compounds of formulas 1a and 1b are referred to below as “ectoin or ectoin derivatives”.

Ectoin and the ectoin derivatives are low-molecular, cyclic amino-acid derivatives obtained from various halophilic microorganisms. Both ectoin and ectoin derivatives have the advantage that they do not interfere with cell metabolism. Ectoin and ectoin derivatives have already been described in DE 43 42 560 as moisturizing agents for use in cosmetic products.

The compounds used in the present invention can be present in topical formulations in the form of optical isomers, diastereoisomers, racemates, zwitterions, cations, or a mixture thereof.

The compounds used in the present invention are preferably those in which R¹ denotes H or CH₃, R² denotes H or COOH, R³ and R⁴ independently denote H or OH, and n is 2. Of the compounds used in the present invention, (S)-1,4,5,6-tetrahydro-2-methyl-4-pyrimidinecarboxylic acid (ectoin) and (S,S)-1,4,5,6-tetrahydro-5-hydroxy-2-methyl-4-pyrimidinecarboxylic acid (hydroxylectoin) are particularly preferred.

By the term “amino acid” we mean the stereoisomeric forms, eg, D and L forms of the following compounds: alanine, β-alanine, arginine, asparagine, aspartic acid, cysteine, glutamine, glutamic acid, glycine, histidine, isoleucine, leucine, lysine, methionine, phenylalanine, serine, threonine, tryptophane, tyrosine, valine, γ-aminobutyrate, Nε-acetyllysine, Nδ-acetylornithine, Nγ-acetyldiaminobutyrate, and Nα-acetyldiaminobutyrate. L-amino acids are preferred.

Amino-acid residues are derived from the corresponding amino acids.

The residues of the following amino acids are preferred: alanine, β-alanine, asparagine, aspartic acid, glutamine, glutamic acid, glycine, serine, threonine, valine, γ-aminobutyrate, Nε-acetyllysine, Nδacetylornithine, Nγ-acetyldiaminobutyrate, and Nα-acetyldiaminobutyrate.

The dipeptide and tripeptide residues are acid amides by chemical nature and decompose under hydrolysis to form two or three amino acids. The amino acids in the dipeptide and tripeptide residues are bonded to each other by amide linkages. Preferred dipeptide and tripeptide residues are based on the preferred amino acids.

The alkyl groups comprise the methyl group CH₃, the ethyl group C₂H₅, the propyl groups CH₂CH₂CH₃ and CH(CH₃)₂, and the butyl groups CH₂CH₂CH₂CH₃, H₃CCHCH₂CH₃, CH₂CH(CH₃)₂, and C(CH₃)₃. The preferred alkyl group is the methyl group.

Preferred physiologically acceptable salts of the compounds used in the present invention are, for example, alkali salts, alkaline earth metal salts, or ammonium salts, such as Na, K, Mg, or Ca salts, and also salts which are derived from the organic bases triethylamine or tris(2-hydroxyethyl)amine. Other preferred physiologically acceptable salts of the compounds used in the present invention are obtained by reaction with inorganic acids, such as hydrochloric acid, sulfuric acid, and phosphoric acid, or with organic carboxylic or sulfonic acids, such as acetic acid, citric acid, benzoic acid, maleic acid, fumaric acid, tartaric acid, and p-toluene-sulfonic acid.

Compounds of formulas 1a and 1b in which base groups and acid groups, such as carboxyl or amino groups, are present in equal number, form internal salts.

The production of the compounds used in the present invention is described in DE 43 42 560. (S)-1,4,5,6-tetrahydro-2-methyl-4-pyrimidinecarboxylic acid or (S,S)-1,4,5,6-tetrahydro-5-hydroxy-2-methyl-4-pyrimidinecarboxylic acid may alternatively be obtained microbiologically (Severin et al, J. Gen. Microb. 138 (1992) 1629-1638).

According to the invention, ectoin or ectoin derivatives are usually employed in the form of a topical composition.

The production of the topical composition is effected by converting at least one of the compounds used in the present invention, optionally together with adjuvants and/or vehicles, to a suitable formulation form. The adjuvants and vehicles are selected from the group comprising vehicles, preservatives, and other conventional adjuvants.

The topical composition based on at least one compound used in the present invention is applied externally to the skin or skin adnexa.

As examples of suitable administration forms there may be mentioned: solutions, suspensions, emulsions, pastes, ointments, gels, creams, lotions, powders, soaps, surfactant-containing cleaning preparations, oils, and sprays. In addition to one or more compounds used in the present invention, any conventional vehicles, adjuvants, and, optionally, other active substances may be added to the composition.

Preferred adjuvants are selected from the group comprising preservative agents, antioxidants, stabilizing agents, solutizers, vitamins, coloring agents, and odor improvers.

Ointments, pastes, creams, and gels may contain, in addition to one or more compounds used in the present invention, conventional vehicles, eg, animal and vegetable fats, waxes, paraffin waxes, starch, gum traganth, cellulose derivatives, polyethylene glycols, silicones, bentonites, silicic acid, talcum powder, zinc oxide, or mixtures of these materials.

Powders and sprays may contain, in addition to one or more compounds used in the present invention, conventional vehicles, eg, lactose, talcum powder, silicic acid, aluminum hydroxide, calcium silicate, polyamide powder, or mixtures of these materials. Sprays can additionally contain conventional aerosol propellants, eg, chlorofluorocarbons, propane/butane, or dimethyl ether.

Solutions and emulsions may contain, in addition to one or more compounds used in the present invention, conventional vehicles, such as solvents, solutizers, and emulsifiers, eg, water, ethanol, isopropanol, ethyl carbonate, ethyl acetate, benzyl alcohol, benzyl benzoate, propylene glycol, 1,3-butyl glycol, oils, particularly cotton-seed oils, peanut oil, maize germ oil, olive oil, castor oil, and sesame oil, glycerin fatty acid esters, polyethylene glycols, fatty acid esters of sorbitan, or mixtures of these materials.

Suspensions may contain, in addition to one or more compounds used in the present invention, conventional vehicles, such as liquid diluents, eg, water, ethanol, or propylene glycol, suspending agents, eg, ethoxylated isostearyl alcohols, polyoxyethylene sorbitol ester, and polyoxyethylene sorbitan ester, microcrystalline cellulose, aluminum metahydroxide, bentonite, agar-agar, gum traganth, or mixtures of these materials.

Soaps may contain, in addition to one or more compounds used in the present invention, conventional vehicles, such as alkali-metal salts of fatty acids, salts of fatty acid half-esters, fatty acid albumin hydrolysates, isothionates, lanoline, fatty alcohol, plant oils, plant extracts, glycerin, sugar, or mixtures of these materials.

Surfactant-containing cleaning products may contain, in addition to one or more compounds used in the present invention, conventional vehicles, such as salts of fatty alkyl sulphates, fatty alcohol ether sulfates, sulfosuccinic acid half-esters, fatty acid albumin hydrolysates, isothionates, imidazolinium derivatives, methyl taurates, sarcosinates, fatty amide ether sulfates, alkyl amidobetaines, fatty alcohols, fatty acid glycerides, fatty acid diethanolamides, vegetable and synthetic oils, lanoline derivatives, ethoxylated glycerin fatty acid esters, or mixtures of these materials.

Face oils and body oils may contain, in addition to one or more compounds used in the present invention, the usual vehicles, such as synthetic oils, for example fatty acid esters, fatty alcohols, silicone oils, and natural oils, such as plant oils and oily plant extracts, paraffin oils, lanoline oils, or mixtures of these materials.

Other, typically cosmetic, administration forms are lipsticks, lip-care sticks, mascara, eyeliners, eye-shadow, rouge, powder make-up, emulsion make-up, wax make-up, sun-screening preparations, pre-sun preparations, and after-sun preparations.

At least one compound used in the present invention is present in the topical composition in a concentration of preferably from 0.0001 to 50 wt %, more preferably from 0.001 to 10 wt %, and most preferably from 0.1 to 1 wt %, based on the composition.

Preferably, at least one antioxidant and/or UV filter is used in addition to ectoin or the ectoin derivatives.

According to the invention, the antioxidants disclosed in the technical literature may be used, for example, flavonoids, coumaranones, amino acids (eg, glycine, histidine, tyrosine, tryptophane) and derivatives thereof, imidazoles, (eg, urocanic acid) and derivatives thereof, peptides, such as D,L-carnosine, D-carnosine, L-carnosine, and derivatives thereof (eg, anserine), carotenoids, carotenes (eg, α-carotene, β-carotene, lycopene) and derivatives thereof, chlorogenic acid and derivatives thereof, lipoic acid and derivatives thereof (eg, dihydrolipoic acid), aurous thioglucose, propyl thiouracil and other thiols (eg, thioredoxin, glutathione, cysteine, cystine, cystamine and their glycosyl, N-acetyl, methyl, ethyl, propyl, amyl, butyl, and lauryl, palmitoyl, oleyl, γ-linoleyl, cholesteryl, and glycerin esters) and also their salts, dilauryl thiodipropionate, distearyl thiodipropionate, thiodipropionic acid and derivatives thereof (esters, ethers, peptides, lipids, nucleotides, nucleosides, and salts) and also sulfoximine compounds (eg, buthioninsulfoximines, homocysteinesulfoximine, buthioninsulfones, penta-, hexa-, or hepta-thioninesulfoximine), further (metallic) chelating agents (eg, α-hydroxyl fatty acids, palmitic acid, phytic acid, lactoferrin), α-hydroxyl acid (eg, citric acid, lactic acid, malic acid), humic acid, bile acid, biliary extracts, bilirubin, biliverdin, EDTA, EGTA and derivatives thereof, unsaturated fatty acids and derivatives thereof, vitamin C and derivatives (eg, ascorbyl palmitate, magnesium ascorbyl phosphate, ascorbyl acetate) and also coniferyl benzoate of benzoin, rutinic acid and derivatives thereof, α-glycosyl rutin, ferulic acid, furfurglucitol, carnosine, butyl hydroxyltoluene (BHT), butylated hydroxyanisole, nordohydroguaiaretic acid, and trihydroxybutyrophenone, uric acid and derivatives thereof, mannose and derivatives thereof, zinc and derivatives thereof (eg, ZnO, ZnSO₄), selenium and derivatives thereof (eg, selenomethionine), and stilbenes and derivatives thereof (eg, stilbene oxide and transstilbene oxide).

Mixtures of antioxidants are equally suitable. Known and commercial mixtures are, for example, mixtures containing as active constituents lecithin, L-(+)-ascorbyl palmitate, and citric acid (eg, Oxynex® AP), natural tocopherols, L-(+)-ascorbyl palmitate, L-(+)-ascorbic acid, and citric acid (eg, Oxynex® K LIQUID), tocopherol extracts from natural sources, L-(+)-ascorbyl palmitate, L-(+)-ascorbic acid, and citric acid (eg, Oxynex® L LIQUID), D,L-α-tocopherol, L-(+)-ascorbyl palmitate, citric acid and lecithin (eg, Oxynex® LM) or butyl hydroxytoluene (BHT), L-(+)-ascorbyl palmitate, and citric acid (eg, Oxynex® 2004).

In a preferred embodiment of the invention, the antioxidant used is butyl hydroxytoluene. In another preferred embodiment, the antioxidant used comprises one or more compounds selected from the group comprising flavonoids and/or coumaranones.

Flavanoids are taken to mean glycosides of flavanones, flavones, 3-hydroxy-flavones (=flavanols), aurones, isoflavones, and rotenoids (Römpp Chemie Lexikon, Vol. 9, 1993). However, for the purposes of the present invention, they are also taken to include the aglycons, ie aglycosuric components, and the derivatives of said flavonoids and aglycons. For the purposes of the present invention, coumaranones are also taken to include their derivatives.

Preferred flavonoids are derived from flavanones, flavones, 3-hydroxyflavones, aurones, and isoflavones, particularly from flavanones, flavones, 3-hydroxyflavones, and aurones.

The flavanones are characterized by the following basic structure:

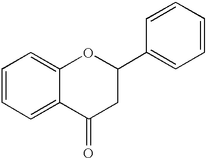

The flavones are characterized by the following basic structure:

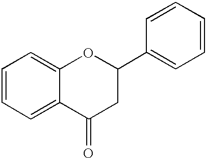

The 3-hydsroxyflavones (flaonols) are characterized by the following basic structure:

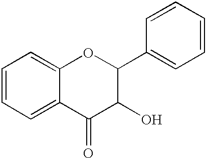

The isoflavones are characterized by the following basic structure:

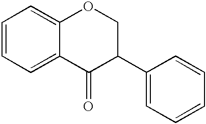

The aurones are characterized by the following basic structure:

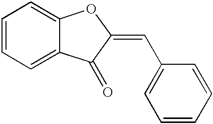

The coumaranones are characterized by the following basic structure:

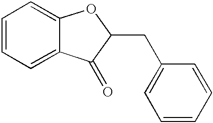

Preferably, the flavonoids and coumaranones are selected from the group comprising the compounds of formula (I):

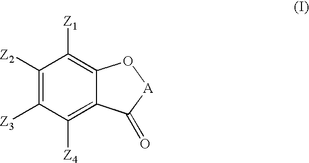

in which

Z₁ to Z₄ independently denote H, OH, alkoxy, hydroxyalkoxy, and mono- or oligo-glycoside groups, in which the alkoxy and hydroxyalkoxy groups can be branched or unbranched and can exhibit from 1 to 18 carbons and in which sulfate or phosphate may also be bonded to the hydroxyl groups in said groups,

A is selected from the group comprising the partial forms (IA), (IB) and (IC)

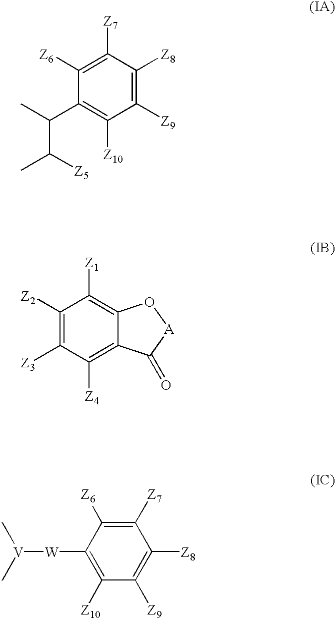

in which

Z₅ denotes H, OH, or OR,

R denotes a monoglucoside or oligoglycoside group,

Z₆ to Z₁₀ have the meanings given above for Z₁ to Z₄, or denote

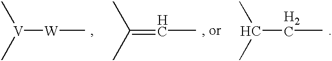

The alkoxyl groups are preferably linear and possess from 1 to 12 and preferably. from 1 to 8 carbons. Thus these groups conform to the formula —O—(CH₂)ₘ—H, in which m is 1, 2, 3, 4, 5, 6, 7, or 8, and, in particular, 1 to 5.

The hydroxyalkoxy groups are preferably linear and possess from 2 to 12, and preferably from 2 to 8 carbon atoms. Thus these groups conform to the formula —O—(CH₂)ₙ—OH, in which n is 2, 3, 4, 5, 6, 7, or 8, preferably from 2 to 5, and more preferably 2.

The monoglycoside and oligoglycoside groups are preferably composed of from 1 to 3 glycoside units. Preferably, these units are selected from the group comprising hexosyl residues, and particularly rhamnosyl residues and glucosyl residues. Alternatively, other hexosyl residues, for example, allosyl, altrosyl, galactosyl, gulosyl, idosyl, mannosyl, and talosyl, may be used to advantage, if desired. Alternatively, it may be advantageous to use pentosyl residues in the present invention.

In a preferred embodiment, the meanings of the variants are as follows:

Z₁ and Z₃ each denote H,

Z₂ and Z₄ are other than H, and denote, in particular, OH, methoxy, ethoxy or 2-hydroxyethoxy,

Z₅ denotes H, OH, or a glycoside residue composed of from 1 to 3, and preferably 1 or 2, glycoside units,

Z₆, Z₉ and Z₁₀ each denote H, and

Z₇ and Z₈ are other than H, and preferably denote OH, methoxy, ethoxy, or 2-hydroxyethoxy.

In another preferred embodiment, particularly when the water solubility of the flavonoids and coumaranones is to be raised, a sulfate or phosphate group is bonded to the hydroxyl group. Suitable counterions are, for example, the ions of the alkali metals or alkaline-earth metals, these being selected from the group comprising, for example, sodium and potassium.

In another preferred embodiment, the flavonoids are selected from the group comprising the following compounds: 4,6,3′,4′-tetrahydroxyaurone, quercetin, rutin, isoquercetin, anthocyanidin (cyanidin), eriodictyol, taxifolin, luteolin, trishydroxyethylquercetin (troxequercetin), trishydroxyethylrutin (troxerutin), trishydroxyethylisoquercetin (troxeisoquercetin), and trishydroxyethylluteolin (troxeluteolin), and their sulfates and phosphates.

Of the flavonoids, rutin and troxerutin are especially preferred, and particular preference is given to troxerutin.

Among the coumaranones, preference is given to 4,6,3′,4′-tetrahydroxybenzyl-coumaranone-3.

According to the invention, the antioxidants are used in the topical composition in conventional concentrations.

Furthermore, UV filters disclosed in the technical literature can be used in the present invention.

Suitable organic UV filters are all UVa and UVb filters known to the person skilled in the Art. For both UV ranges there are many substances which have been disclosed in the technical literature and which have been used with success, eg,

benzylidenecamphor derivatives, such as

3-(4′-methylbenzylidene)-dl-camphor (eg, Eusolex® 6300),

3-benzylidenecamphor (eg, Mexoryl® SD),

polymers of N-{(2 and 4)-[(-3-oxoborn4-ylidene)methyl]benzyl}acrylamide (eg, Mexoryl® SW),

N,N,N-trimethyl-4-(2-oxoborn-3-ylidenemethyl)anilinium methylsulfate (eg, Mexoryl® SK) or

α-(2-oxobornyl-3-ylidene)toluene-4-sulfonic acid (eg, Mexoryl® SL),

benzoyl- or dibenzoyl-methanes, such as

1-(4-tert-butylphenyl)-3-(4-methoxyphenyl)propane-1,3-dione (eg, Eusolex® 9020) or

4-isopropyldibenzoylmethane (eg, Eusolex® 8020),

benzophenones, such as

2-hydroxy-4-methoxybenzophenone (eg, Eusolex® 4360) or

2-hydroxy4-methoxybenzophenone-5-sulfonic acid and the sodium salt thereof (eg, Uvinul® MS 40),

methoxycinnamates; such as

2-ethylhexyl p-methoxycinnamate (eg, Eusolex® 2292),

isopentyl p-methoxycinnamate, eg, as a mixture of the isomers (eg, Neo Heliopan® E 1000),

salicylate derivatives, such as

2-ethylhexyl salicylate (eg, Eusolex® OS),

4-isopropylbenzyl salicylate (eg, Megasol® ) or

3,3,5-trimethylcyclohexyl salicylate (eg, Eusolex® HMS),

4-aminobenzoic acid and derivatives thereof, such as

4-aminobenzoic acid,

2-ethylhexyl 4-(dimethylamino)benzoate (eg, Eusolex® 6007),

ethoxylated ethyl 4-aminobenzoate (eg, Uvinul® P25),

and other substances, such as

2-ethylhexyl 2-cyano-3,3-diphenylacrylate (eg, Eusolex® OCR),

2-phenylbenzimidazol-5-sulfonic acid, and the potassium, sodium, and tri-ethanolamine salts thereof (eg, Eusolex® 232)

3,3′-(1,4-phenylenedimethylene)-bis(7,7-dimethyl-2-oxobicyclo[2.2.1]heptyl-1-methanesulfonic acid, and the salts thereof (eg, Mexoryl® SX), and

2,4,6-trianilino-(p-carbo-2′-ethylhexyl-1′-oxo)-1,3,5-triazine (eg, Uvinul® T 150).

These organic UV filters are usually employed in the topical composition used in the present invention in a concentration of from 0.5 to 10 wt %, and preferably from 1 to 8 wt %.

Other suitable organic UV filters are, for example,

2-(2H-benzotriazol-2-yl)-4methyl-6-(2-methyl-3-(1,3,3,3-tetramethyl-1-(trimethylsilyloxy)disiloxanyl)propyl)phenol (eg, Silatrizole®),

4,4′-[(6-[4-((1,1-dimethylethyl)aminocarbonyl)phenylamino]-1,3,5-triazin-2,4-diyl)diimino]-bis(2-ethylhexyl benzoate) (eg, Uvasorb® HEB),

α-(trimethylsilyl)-ω-[trimethylsilyl)oxy]poly[oxy(dimethyl] [and ca 6% of methyl[2-[p-[2,2-bis(ethoxycarbonyl]vinyl]phenoxy]-1-methylenethyl] and ca 1.5% of methyl[3-[p-[2,2-bis(ethoxycarbonyl)vinyl)phenoxy)propenyl) and from 0.1 to 0.4% of (methylhydrogen]silylene]](n≈60) (eg, Parsol® SLX,

2,2′-methylene-bis(6-(2-H-benzotriazolyl-2)-4-(1,1,3,3-tetramethylbutyl)-phenol (eg, Tinosorb® M),

2,2′-(1,4-phenylene)-bis(1H-benzimidazol-4,6-disulfonic acid and the monosodium salt thereof,

2,2′-(1,4-phenylene)-bis(1H-benzimidazol-5-sulfonic acid and the monosodium salt thereof,

2,2′-(1,4-phenylene)-bis(1H-benzimidazol-5-sulfonic acid and the monopotassium salt thereof, and

2,4-bis{[4-(2-ethylhexyloxy)-2-hydroxyl]phenyl}-6-(4-methoxyphenyl)-1,3,5-triazine (eg, Tinosorb® S).

These organic filters are usually employed in the topical composition used in the present invention in a concentration of from 0.5 to 20 wt %, and preferably from 1 to 15 wt %.

Conceivable inorganic UV filters are those selected from the group comprising titanium dioxides, eg, coated titanium dioxide (eg, Eusolex® T 2000 or Eusolex® T-Aqua), zinc oxides (eg, Sachtotec®), iron oxides or, alternatively, cerium oxides. These inorganic UV filters are usually employed in the topical composition used in the present invention in a concentration of from 0.5 to 20 wt %, and preferably from 2 to 10 wt %.

Preferred UV filters are zinc oxide, titanium dioxide, 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazol-5-sulfonic acid, and the potassium, sodium, and triethanolamine salts thereof.

Particularly preferred UV filters are zinc oxide and titanium dioxide.

If titanium dioxide is used in the present invention, there are preferably used, in addition to titanium dioxide, one or more further UV filters, selected from the group comprising 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazol-5-sulfonic acid, and the potassium, sodium, and triethanolamine salts thereof.

It is particularly preferred to use, in addition to titanium dioxide, the UV filters 2-hydroxy4-methoxybenzophenone and/or 2-ethylhexyl p-methoxycinnamate.

In the present invention, ectoin or ectoin derivatives can be used for the prophylaxis and/or treatment of UV-induced immunosuppression. The use of ectoin or ectoin derivatives as proposed in the present invention effects protection of Langerhans cells in the skin. Furthermore, the dentritic morphology of the Langerhans cells and their ability to present antigen is maintained by the use of ectoin or ectoin derivatives as proposed in the present invention. The overall result is that UV-induced immunosuppression can be effectively avoided.

The following examples illustrate the present invention. All compounds or ingredients that can be used in the cosmetic formulations are either known and commercially available or can be synthesized by known methods.

The INCI names of the starting materials used are as follows (the INCI names are, by way of definition, always stated in the English language):

| STARTING MATERIAL | INCI-NAME |
|---|---|
| Glycerin | Glycerin |
| Paraffin, liquid | Mineral Oil (Paraffinum Liquidum) |
| Mirasil CM 5 | Cyclomethicone |
| Arlacel 165 | Glyceryl Stearate, PEG-100 Stearate |
| Germaben II | Propylene Glycol, Diazolidinyl Urea, Methyl- |
| | paraben, Propylparaben |
| Isopropyl myristate | Isopropyl Myristate |
| water, demineralized | Aqua |
| Stearic acid | Stearic Acid |

### EXAMPLE 1

Hair Tonic Containing Ectoin

| Starting material | | INCI-Name | Wt % |
|---|---|---|---|
| MERCARE ® Biotin | (1) | Biotin | 0.05 |
| Art. Nr. 130220 | | | |
| MERCARE ® Ectoin | | (Ectoin) | 1.00 |
| Art. Nr. 130200 | | | |
| Octopirox | (2) | Piroctone Olamine | 0.10 |
| D(+)Pantothenyl alcohol | (3) | Panthenol | 0.30 |
| (Art. Nr. 501375) | | | |
| Salicylic acid | (1) | Salicylic Acid | 0.10 |
| (Art. Nr. 100631) | | | |
| N-Cetyl-N,N,N-trimethyl- | (1) | Cetrimonium Bromide | 0.10 |
| ammonium bromide | | | |
| (Art. Nr. 102343) | | | |
| Dragoplant Hamamelis | (4) | Aqua, Alcohol Dentat., | 1.00 |
| | | Hamamelis Virginiana | |
| Isopropyl alcohol | (1) | Isopropyl Alcohol | 45.00 |
| (Art.-Nr. 100995) | | | |
| Demin. water | | Aqua | ad 100 |

Preparation:

Biotin was dissolved in water und isopropyl alcohol. Ectoin was then dissolved, and the remaining starting materials were added with stirring.

Sources of Supply:

(1) Merck KGaA

(2) Hoechst

(2) BASF

(3) Dragoco

### EXAMPLE 2

2 in 1 Shampoo

| Starting material | | INCI-Name | Wt % |
|---|---|---|---|
| Jaguar C-162 | (2) | Hydroxypropyl Guar | 0.20 |
| | | Hydroxypropyltrimonium | |
| | | Chloride | |
| Miranol Ultra C32 | (2) | Sodium Cocoamphoacetat | 10.00 |
| Texapon NSO | (3) | Sodium Laureth Sulfate | 32.00 |
| Nicotinamide (Vitamin B3) | (1) | Niacinamide | 0.10 |
| (Art. Nr. 130179) | | | |
| (D+)-Biotin (Vitamin H) | (1) | Biotin | 0.05 |
| (Art. Nr. 130220) | | | |
| MERCARE ® Ectoin | (1) | (Ectoin) | 1.00 |
| (Art. Nr. 130200 | | | |
| D-Panthenol | (4) | Panthenol | 0.50 |
| Sodium chloride | (1) | Sodium Chloride | 1.0 |
| (Art. Nr. 106400) | | | |
| Perfume | | Parfum | |
| Preservative | | | q.s. |
| Citric acid | (1) | Citric Acid | q.s. |
| (Art. Nr. 130137) | | | |
| Demin. water | | Aqua | ad 100 |

Preparation:

The pearlescent pigment was dispersed in the water/propanol mixture of phase A and the Carbopol was disseminated with stirring. Following complete dissolution the predissolved phase B was slowly stirred in.

Comments:

Recommended pearlescent pigments are interference pigments, silver pigments, gold pigments, and iron oxide pigments.

Sources of Supply:

(1) Merck KGaA

(2) BF Goodrich GmbH

(3) BASF AG

(4) ISP Global Technologies

### EXAMPLE 4

Syndet Soap

| Starting material | | INCI-Name | Wt % |
|---|---|---|---|
| Zetasap 813 A | (2) | Disodium Lauryl Sulfosuccinate, So- | 90.0 |
| | | dium Cocoyl Isothionate, | |
| | | Cetearyl Alcohol, Corn Starch, Glyc- | |
| | | eryl Stearate, Paraffin, | |
| | | Titanium Dioxide | |
| Ectoin | (1) | (Ectoin) | 1.00 |
| (Art. No. 130200) | | | |
| Perfume | | Parfum | 1.00 |
| Demin. water | | Aqua (Water) | 8.00 |

Sources of Supply:

(1) Merck KGaA

(2) Zschimmer & Schwarz

### EXAMPLE 5

Shower Gel

| Starting material | Art. No. | | INCI-Name | Wt % |
|---|---|---|---|---|
| A | | | | |
| Timiron Splendid | 1.17477 | (1) | CI 77891 (Titanium Diox- | 0.10 |
| Green | | | ide), Mica, Silica | |
| Keltrol T | | (2) | Xanthan Gum | 0.75 |
| Water, demineral- | | | Aqua (Water) | 62.10 |
| ized | | | | |
| B | | | | |
| Plantacare 2000 | | (3) | Decyl Glucoside | 20.00 |
| Texapon ASV | | (3) | Magnesium Oleth Sulfate, | 0.65 |
| | | | Sodium Oleth Sulfate, | |
| | | | Magnesium Laureth-8 Sulfate, | |
| | | | Sodium Laureth-8 Sulfate, | |
| | | | Magnesium Laureth Sulfate, | |
| | | | Sodium Laureth Sulfate | |
| Bronidox L | | (3) | Propylene Glycol 5-Bromo- | 0.20 |
| | | | 5-nitro-1,3-dioxane | |
| Perfume oil Everest | | (4) | Parfum | 0.05 |
| 79658 SB | | | | |
| MERCARE ® | 130200 | (1) | (Ectoin) | 1.00 |
| Ectoin | | | | |
| C | | | | |
| Citric acid mono- | 130137 | (1) | Citric Acid | 0.15 |
| hydate | | | | |
| Water, demineral- | | | Aqua (Water) | 10.00 |
| ized | | | | |

Preparation:

To create phase A, the pigment was stirred into the water. Keltrol T was slowly disseminated with stirring, and stirring was continued until it was dissolved. Phases B and C were successively added, and slow stirring was continued until all ingredients were homogeneously distributed.

Sources of Supply:

(1) Merck KGaA

(2) Kelco

(3) Cognis GmbH

(4) Haanmann & Reimer GmbH

### EXAMPLE 6

Baby Powder

| Starting material | Art. No. | | INCI-Name | Wt % |
|---|---|---|---|---|
| A | | | | |
| IR 3535 TM | 111887 | (1) | Ethylbutylacetylamino- | 4.00 |
| | | | propionate | |
| B | | | | |
| Magnesium carbonate | 105827 | (1) | Magnesium Carbonate | 10.00 |
| hydroxide | | | Hydroxdie | |
| Dry Flo PC | | (2) | Aluminium Starch | 86.00 |
| | | | Octenylsuccinate | |
| MERCARE ® Ectoin | 130200 | (1) | (Ectoin) | 1.00 |

Preparation:

Phase B was used as initial batch and mixed with a propeller-type stirrer. Phase A was added dropwise with stirring.

Sources of Supply:

(1) Merck KGaA

(2) National Starch & Chemical

### EXAMPLE 7

O/W After-Sun Lotion

| Starting material | Art. No. | | INCI-Name | Wt % |
|---|---|---|---|---|
| A | | | | |
| MERCARE ® Bisabolol | 130170 | (1) | Bisabolol | 0.30 |
| Montanov 68 | | (2) | Cetearyl Alcohol, | 4.00 |
| | | | Cetearyl Glucoside | |
| Miglyol 812, neutral oil | | (3) | Caprylic/Capric | 12.00 |
| | | | Triglyceride | |
| Mirasil CM5 | | (4) | Cyclomethicone | 2.00 |
| Mirasil DM 350 | | (4) | Dimethicone | 1.00 |
| B | | | | |
| Water, demineralized | | | Aqua (Water) | 77.20 |
| Glycerin (87% high-grade) | 104091 | (1) | Glycerin | 3.00 |
| Preservative | | | | q.s. |
| MERCARE ® Ectoin | 130200 | (1) | (Ectoin) | 1.00 |
| C | | | | |
| Rhodicare-S | | (4) | Xanthan Gum | 0.50 |

Preparation:

Phases A and B were separately heated to 75° C., phase C was slowly added to phase B at 75° C. with stirring, and the mixture was stirred until homogeneous. Phase A was then added to the mixture B/C and homogenized. With stirring, the resulting mixture was cooled to room temperature.

Sources of Supply:

(1) Merck KGaA

(2) Seppic

(3) Hüls AG

(4) Rhodia GmbH

### EXAMPLE 8

Sunscreen Lotion (W/O)

| Starting material | Art. No. | | INCI-Name | Wt % |
|---|---|---|---|---|
| A | | | | |
| Eusolex 8300 | 105385 | (1) | 4-Methylbenzylidene | 4.00 |
| | | | Camphor | |
| Eusolex 2292 | 105382 | (1) | Octylmethoxycinnamate, | 7.00 |
| | | | BHT | |
| Abil WE 09 | | (2) | Polyglyceryl-4-isostea- | 5.00 |
| | | | rate, Cetyl Dimethicone | |
| | | | Copolyol, Hexyl Laurate | |
| Jojoba oil | | (3) | Buxus Chinensis (Jojoba | 3.00 |
| | | | Oil) | |
| Cetiol V | | (4) | Decyloleate | 3.00 |
| Prisorine 2021 | | (5) | Isopropyl Isostearate | 2.00 |
| Paracera M | | (6) | Microwax | 1.00 |
| Miglyol 812, Neutralöl | | (7) | Caprylic/Capric Triglyc- | 3.00 |
| | | | eride | |
| Propyl 4-hydroxyben- | 1.07427 | (1) | Propylparaben | 0.05 |
| zoate | | | | |
| B | | | | |
| Eusolex T-Aqua | 105401 | (1) | Aqua (Water), | 16.00 |
| | | | Titanium Dioxide, | |
| | | | Alumina, Sodium Meta- | |
| | | | phosphate, Phenoxy- | |
| | | | ethanol, Sodium Methyl- | |
| | | | paraben | |
| Glycerin (87% high- | 104091 | (1) | Glycerin | 2.00 |
| grade) | | | | |
| Sodium chloride | 106400 | (1) | Sodium Chloride | 0.40 |
| MERCARE ® Ectoin | 130200 | (1) | (Ectoin) | 1.00 |
| Water, demineralized | | | Aqua (Water) | 53.40 |
| Methyl 4-hydroxyben- | 106757 | (1) | Methylparaben | 0.15 |
| zoate | | | | |

Preparation:

Phase B was heated to 80 ° C. and phase A to 75 ° C. Phase B was slowly stirred into phase A. The mixture was homogenized and cooled with stirring.

Sources of Supply:

(1) Merck KGaA

(2) Th. Goldschmidt AG

(3) Henry Lamotte GmbH

(4) Cognis GmbH

(5) Unichema Chemie GmbH

(6) Paramelt

(7) Hüls AG

### EXAMPLE 9

Tooth Gel

| Starting material | Art. No. | | INCI-Name | Wt % |
|---|---|---|---|---|
| A | | | | |
| Sodium fluoride | 106441 | (1) | Sodium Fluoride | 0.06 |
| Karion F liquid | 152698 | (1) | Sorbitol | 48.39 |
| Sodium benzoate | 106290 | (1) | Sodium Benzoate | 0.16 |
| Sodium saccharinate | | | | 0.16 |
| MERCARE ® Ectoin | 130200 | (1) | (Ectoin) | 1.00 |
| Water, demineralized | | | Aqua (Water) | 29.12 |
| B | | | | |
| MERCARE ® Olaflur | 111680 | (1) | Olaflur, Propylene | 1.17 |
| | | | Glycol | |
| Bromochlorophene | 1.03281 | (1) | Bromochlorophene | 0.08 |
| Aroma 35049 | | (2) | | 0.78 |
| C | | | | |
| Polyethylene glycol | 807485 | (1) | PEG-8 | 2.34 |
| 400 | | | | |
| Tego Betain ZF | | (3) | Cocamidopropyl Betaine | 3.89 |
| Sicomet Patent Blue | | (4) | | 0.62 |
| (E131), 0.1% in water | | | | |
| D | | | | |
| Sident 12 | | (5) | Silica | 7.40 |
| Sipemat 22 S | | (5) | Hydrated Silica | 5.84 |

Preparation:

Phases A and B were separately premixed. Phase C was heated to 50 ° C. Phases A and B were stirred into phase C, and the mixture was stirred in vacuo. Following slow addition of phase D, the mixture was homogenized in vacuo. Stirring was continued in vacuo until the gel was clear.

Sources of Supply:

(1) Merck KGaA

(2) Crissa Drebing GmbH

(3) Th. Goldschmidt AG

(4) BASF AG

(5) Degussa AG

### EXAMPLE 10

Mouthwash Concentrate

| Starting material | | Wt % |
|---|---|---|
| MERCARE ® Ectoin | (1) | 1.00 |
| N-cetylpyridinium chloride (Art. No. 102340) | (1) | 0.50 |
| Ethanol (96%) (Art. No. 100971) | (1) | 70.00 |
| Peppermint aroma 77526-34 | (2) | 0.15 |
| Water, demineralized | | Ad 100.00 |

Preparation:

The ingredients were stirred together until a clear solution was obtained.

Sources of Supply:

(1) Merck KGaA

(2) Givaudan-Roure, Dortmund

### EXAMPLE 11

Lip Balsam

| Starting material | INCI-Name | | Wt % |
|---|---|---|---|
| Ectoin | (1) | (Ectoin) | 1.00 |
| (Art. No. 130200) | | | |
| Tagat S2 | (2) | PEG-20 Glyceryl Stearate | 10.00 |
| Lanette O | (3) | Cetearyl Alcohol | 20.00 |
| Glycerin (87%) | (1) | Glycerin | 20.00 |
| (Art. No. 104091) | | | |
| Vaseline | (4) | Petrolatum | 35.00 |

Preparation

The ingredients were heated to 75° C. and then cooled to room temperature with stirring.

Sources of Supply:

(1) Merck KGaA

(2) Goldschmidt GmbH

(3) Cognis GmbH

(4) Schümann Sasol

### EXAMPLE 12

Lip Gloss

| Starting material | Art. No. | INCI-Name | | Wt % |
|---|---|---|---|---|
| A | | | | |
| Pearlescent pigments | | (1) | | 10.00 |
| B | | | | |
| Indopol H 100 | | (2) | Polybutene | 59.95 |
| Bentone Gel MIO V | | (3) | Quaternium-18 Hectorite, | 20.00 |
| | | | Propylene Carbonate | |
| | | | Paraffinum Liquidum | |
| | | | (Mineral Oil) | |
| Eutanol | | (4) | Octyldodecanol | 6.00 |
| MERCARE ® | 130180 | (1) | Tocopheryl Acetate | 1.00 |
| Tocopheryl acetate | | | | 1.00 |
| Dow Corning 1403 Fluid | | (5) | Dimethiconol, Dimethicone, | 3.00 |
| Propyl 4-hydroxy-benzoate | 1.07427 | (1) | Propylbarabene | 0.05 |
| C | | | | |
| MERCARE ® Ectoin | | (1) | (Ectoin) | 1.00 |

Preparation:

All of the components of phase B were weighed in together, heated (60-70° C.) and well stirred until a homogeneous composition resulted. Phases B and C were then added, and the mixture was stirred well. The homogeneous mixture was bottled at 50-60° C.

Sources of Supply:

(1) Merck KGaA

(2) Amoco

(3) Rheox

(4) Cognis GmbH

(5) Dow Corning

### EXAMPLE 13

Lip Herpes Cream

| Starting material | INCI-Name | | Wt % |
|---|---|---|---|
| Ectoin | (1) | (Ectoin) | 1.00 |
| (Art. No. 130200) | | | |
| Aciclovir | | (9-[(2-Hydroxyethoxy)- | 5.00 |
| | | methyl]guanin) | |
| Tagat S2 | (2) | PEG-20 Glyceryl Stearate | 10.00 |
| Lanette O | (3) | Cetearyl Alcohol | 20.00 |
| Glycerin (87%) | | Glycerin | 20.00 |
| (Art. No.104091) | | | |
| Vaseline | (4) | Petrolatum | 35.00 |
| Demin. Water | | Aqua (Water) | ad 100 |

Preparation:

All of the ingredients were heated to 75° C. and the mixture was then cooled to room temperature with stirring.

Sources of Supply:

(1) Merck KGaA

(2) Goldschmidt GmbH

(3) Cognis GmbH

(4) Schumann Sasol

### EXAMPLE 14 UND COMPARATIVE EXAMPLE 1

To confirm the efficacy of the ectoin compounds, an O/w emulsion containing ectoin (Example 14) was tested for its cytoprotective action on the number of Langerhans cells in UV-irradiated human skin and compared with an O/w emulsion containing no ectoin (Comparative Example 1). This experiment is illustrated diagrammatically in FIG. 1.

### EXAMPLE 14

The following ingredients are used to create a Cream (O/W) containing Ectoin:

| | | | | Wt % |
|---|---|---|---|---|
| A) | Paraffin. liquid | (Art. No. 107174) | (1) | 8.0 |
| | Isopropyl myristate | (Art. No. 822102) | (1) | 4.0 |
| | Mirasil CM 5 | | (2) | 3.0 |
| | Stearic acid | | (1) | 3.0 |
| | Arlacel 165 V | | (3) | 5.0 |
| B) | Glycerin. 87% | (Art. No. 104091) | (1) | 3.0 |
| | Germaben II | | (4) | 0.5 |
| | Water. demineralized | | | ad 100 |
| C) | Ectoin | | (1) | 1.0 |

Preparation:

Phases A and B are first of all separately heated to 75° C. Phase A is then slowly added to phase B with stirring, and stirring is continued until a homogeneous mixture is formed. Following homogenization of the emulsion, the mixture is cooled to 30° C. with stirring. The mixture is then heated to 35° C. and phase C is added, and the mixture is stirred until homogeneous.

Sources of Supply:

(1) Merck KGaA, Darmstadt

(2) Rhodia

(3) ICI

(4) ISP

(5) Dragoco

### COMPARATIVE EXAMPLE 1

The following ingredients are used to prepare a cream (O/W) not containing Ectoin:

| | | | | Wt % |
|---|---|---|---|---|
| A) | Paraffin. liquid | (Art. No. 107174) | (1) | 8.0 |
| | Isopropyl myristate | (Art. No. 822102) | (1) | 4.0 |
| | Mirasil CM 5 | | (2) | 3.0 |
| | Stearic acid | | (1) | 3.0 |
| | Arlacel 165 V | | (3) | 5.0 |
| B) | Glycerin. 87% | (Art. No. 104091) | (1) | 3.0 |
| | Germaben II | | (4) | 0.5 |
| | water. demineralized | | | ad 100 |

Preparation:

Phases A and B are separately heated to 75 ° C. Phase A is then slowly added to phase B with stirring, and stirring is continued until a homogeneous mixture is formed.

Sources of Supply:

(1) Merck KGaA, Darmstadt

(2) Rhodia

(3) ICI

(4) ISP

(5) Dragoco

The experiment was carried out using the following apparatus and materials:

Apparatus

| Name/Type: | Manufacturer: |
|---|---|
| Sun Simulator SOL 500 with H2 Filter | Dr. Hönle |
| UVB Meter | Dr. Hönle |
| Refrigerator/Deep Freeze | Bosch |
| CO₂ Incubator: Hera cell | Heraeus |
| Vacuum Pump ME2 | Vacuumbrand |
| Vacuum Meter | Vacuumbrand |
| Suction cups | Special design |
| | Skin Investigation and |
| | Technology Hamburg |
| | GmbH |
| Vacuum distributor block | Special design |
| | Skin Investigation and |
| | Technology Hamburg |
| | GmbH |
| Micropipettes 100-1000 μl | Labsystems |
| Mikroscope: CK40 | Olympus |
| Mikroscope: DXC-950 OP | Olympus |
| Pipette control: Pipetus-akku | Hirschmann |
| Vortex: Reaxtop | Heidolph |
| Balance: AR61 | Mettler Toledo |

Materials

| Name/Make: | Manufacturer/Art. No.: |
|---|---|
| Sterile tips for micropipettes | Greiner und Labsystems |
| 24-Hole plates | Greiner |
| Cacodylate buffer | Sigma |
| Formaldehyde | Merck KGaA |
| ATP | Sigma |
| MgSO₄ | Merck KGaA |
| Pb(NO₃)₂ | Merck KGaA |
| Trismal buffer | Sigma |
| NaCl | Sigma |
| Saccharose | Merck KGaA |
| Ammonium sulfide solution | Merck KGaA |
| PBS²⁻ | Gibco BRL |
| Mowiol | Aldrich |

Probands

The group of probands engaged in the experiment consisted of 10 dermatologically healthy probands (5 male and 5 female probands; phototype II-IV). The probands had an average age of 42.1±11.4 years, and their ages ranged from 27.4 to 69.7 years.

Test Areas

Two test areas were marked out on each forearm of each proband, each test area measuring 4×4 cm. No cream was put on the test areas during a period of 48 hours prior to the commencement of testing, and the test areas were not exposed to UV radiation for a week before the commencement of the experiment.

Application

Two of the test areas were treated (ca 2.0 mg/cm²) over a period of 14 days twice a day with the creams described in Example 14 and Comparative Example 1 respectively.

Minimum Erythema Dosage (MED)

In order to ascertain the individual minimum erythema dosage (MED), each proband was irradiated by means of a sun simulator through a stepped light filter on the basis of the dermatological assessment of the phototype of the proband. For this purpose 6 areas in the lateral region of each forearm (outside the test areas) were irradiated at a distance from the source of radiation of from ca 20 to 25 cm using various irradiation times between 2 min 0 s and 18 min 38 s, depending on the phototype of the proband. The irradiation time was progressively increased from area 1 to area 6. Treatment of the test areas with the creams described in Example 14 and Comparative Example 1 respectively was then carried out. In order to determine the MED, the irradiated areas of the probands were assessed visually following the elapse of 24 hours. If, following a period of 24 hours, a slight or fair erythema became perceptible on one or two of the six irradiated areas, the proband was dismissed and asked to meet an appointment 13 days later for irradiation of the reserved test areas. If a test person showed no definite erythema, the lateral region of the other forearm outside the test areas was irradiated through a stepped light filter at increased dosage achieved by reducing the distance from the source of radiation and/or by using a longer irradiation time, and after a further 24 hours the 6 irradiated areas were examined visually to determine the extent of UV-induced skin changes. The evaluation of the UV-induced erythemas was used to determine the individual MED for each test person.

Exposure

14 days after the commencement of the initial application of the creams described in Example 14 and Comparative Example 1 respectively, a final application thereof to the relevant areas was administered ca 20 minutes prior to irradiation of the test areas. The three reserved test areas (one untreated area and the two areas treated with a cream of Example 14 and Comparative Example 1) respectively were then irradiated with a dose of 1.5 MED. The dose was achieved by varying the distance of the forearm from the source of radiation and by adjustment of the exposure time. An untreated, non-irradiated fourth test area remained as control. In order to avoid edge effects, only one test area measuring 4×4 cm was irradiated at a time per forearm.

Preparation of the Suction Blisters

48 hours (±2 hours) after commencement of the preceding exposure, there was applied, by means of strips of plaster, to each test area a suction cup having an internal diameter of 5 mm. The application of a subpressure of from 750 to 700 mbar caused small suction blisters having a diameter of ca 5 mm to form under the suction cups after a period of from 2 to 2.5 hours. The blister skins were carefully removed in a sterile condition by means of fine surgical instruments and collected for a short time in cold physiological buffer solution to await further use (ATPase staining).

ATPase Staining

Analysis was carried out using the blister skins of the suction blisters thus formed. The specimens were subjected to a staining process in 24-hole tissue culture plates, occupying, in each case, a volume of 1.0 mL per cup. In the first step, the specimen (suction blister skin) was briefly rinsed in PBS. The specimen was then incubated in 0.2M cacodylate buffer for 20 minutes at 4 ° C. There followed 3 rinsing steps in 0.9% strength NaCl solution at 4 ° C. (total duration ca 10 min.), after which the specimen was incubated for 30 minutes at 37 ° C. in a staining reagent obtained by mixing 10 mg of adenosine triphosphoric acid in 5 mL of 10% strength MgSO₄ solution with 3 mL of 2% strength Pb(NO₃)₂ solution and 42 mL of 0.2M trismal buffer. This was followed by rinsing twice in 0.9% strength NaCl solution at 4° C. for 5 minutes each time. Subsequent incubation in a 1% strength ammonium sulfide solution caused the formation of a dark-colored PbS deposit. Finally, rinsing (PBS) was carried out a further two times at 4° C. for a total of 5 minutes and the preparation was then transported with a drop of PBS to a microscope slide and covered over. The covering medium was obtained by dissolving 1 g of Mowiol in 3 mL of PBS with heating.

The number of stained Langerhans cells in a specimen was assessed under the microscope, and the cell counts obtained were converted to “Langerhans cells per mm²”. Tables I and II list the resulting cell counts in the relevant test areas of each test person.

Microscopic Assessment

For each test person 4 suction blister specimens were obtained which were analyzed, following ATPase staining, to determine the number of ATPase-positive Langerhans cells per mm². To this end, for each specimen obtained from a test person an arbitrary area at 3 different points was selected and the average taken. The results of this histological evaluation are given in Table I as Langerhans cells per mm². On average, the completely untreated area of all 10 test persons gave a Langerhans cell density of 1073±214 cells per mm². 48 hours after irradiation with a dose of 1.5 MED, the Langerhans cell density in the untreated areas dropped to 623±210 cells per mm². When the areas were treated twice a day with the creams described in Example 14 and Comparative Example 1 respectively for 14 days prior to irradiation, the counts 48 hours after irradiation were, on the average, 844×233 and 680±157 Langerhans cells per mm² respectively (cf Table I).

When the data are regarded in terms of percentages compared with the untreated specimens (cf Table II), it is seen that in the case of the untreated state, irradiation causes the number of Langerhans cells per unit area to decrese to ca 58%. Preliminary treatment with the cream of Comparative Example 1 led to a reduction of Langerhans cells to ca 64% of the original value and preliminary treatment with the cream of Example 14 to a reduction to ca 78%.

Treatment with the cream of Comparative Example 1 led to no significant reduction in the UV-induced decrease of Langerhans cells (by comparison, with untreated, irradiated specimens). Preliminary treatment with the ectoin-containing cream of Example 14 led to a highly significant reduction of the UV-induced depletion of epidermal Langerhans cells (by comparison with untreated, irradiated skin specimens). The use of ectoin or ectoin derivatives as proposed in the present invention in the form of an O/W emulsion showed, within the scope of the experimental conditions used, significant cytoprotective properties as regards the UV-induced depletion of epidermal Langerhans cells.

TABLE I
Langerhans Cells/mm²*

| | Treatment | | | |
|---|---|---|---|---|
| | | untreated/ | Comp. Example 1/ | Example 14/ |
| Proband | untreated | irradiated | irradiated | irradiated |
| 1 | 981.7 | 563.0 | 648.7 | 824.7 |
| 2 | 1275.7 | 984.7 | 848.3 | 1120.0 |
| 3 | 1284.7 | 540.7 | 620.0 | 800.0 |
| 4 | 1215.3 | 911.7 | 803.0 | 1106.3 |
| 5 | 1241.0 | 800.3 | 851.3 | 1168.7 |
| 6 | 1258.0 | 420.0 | 624.0 | 790.3 |
| 7 | 780.3 | 367.7 | 328.7 | 584.7 |
| 8 | 1092.7 | 651.0 | 788.7 | 906.0 |
| 9 | 842.3 | 466.3 | 602.3 | 535.0 |
| 10 | 761.3 | 525.3 | 681.0 | 588.0 |
| Mean | 1073.3 | 623.1 | 679.6 | 842.4 |
| SD | 214.3 | 210.1 | 157.0 | 232.8 |

*Mean of 3 assessments

TABELLE II
Langerhans Cells/mm² as Percentage of the Untreated State

Treatment

| | | untreated/ | Comp. Example 1/ | Example 14/ |
|---|---|---|---|---|
| Proband | untreated | irradiated | Irradiated | Irradiated |
| 1 | 100.0 | 57.4 | 66.1 | 84.0 |
| 2 | 100.0 | 77.2 | 66.5 | 87.8 |
| 3 | 100.0 | 42.1 | 48.3 | 62.3 |
| 4 | 100.0 | 75.0 | 66.1 | 91.0 |
| 5 | 100.0 | 64.5 | 68.6 | 94.2 |
| 6 | 100.0 | 33.4 | 49.6 | 62.8 |
| 7 | 100.0 | 47.1 | 42.1 | 74.9 |
| 8 | 100.0 | 59.6 | 72.2 | 82.9 |
| 9 | 100.0 | 55.4 | 71.5 | 63.5 |
| 10 | 100.0 | 69.0 | 89.4 | 77.2 |
| Mean | 100.0 | 58.1 | 64.0 | 78.1 |
| SD | 0.0 | 14.2 | 13.9 | 11.9 |

### EXAMPLE 15

Hydrogel Containing Ectoin

| Starting material | Art. No. | INCI-Name | | Wt % |
|---|---|---|---|---|
| A | | | | |
| TIMIRON ®Splendid Gold | 117474 | (1) | CI 77891 (Titanium Dioxide), | 0.10 |
| | | | Mica, Silica | |
| Carbopol Ultrez 10 | | (2) | Carbomer | 0.40 |
| Water, demineralized | | | Aqua (Water) | 67.70 |
| B | | | | |
| RonaCare ™ Ectoin | 130200 | (1) | Ectoin | 1.00 |
| Tris(hydroxymethyl)amino- | 130132 | (1) | Tromethamine | 0.60 |
| Methane | | | | |
| Germaben II | | (3) | Propylene Glycol, Diazolidinyl, | 0.20 |
| | | | Urea, Methylparaben, Propylparaben | |
| Water, demineralized | | | Aqua (Water) | 10.00 |
| C | | | | |
| Lubrajel DV | | (4) | Propylene Glycol, Polyglyceryl | 18.00 |
| | | | Methacrylate | |
| D | | (4) | PVM/MA Copolymer, | |
| Lubrajel Oil | | | Proplyene | |
| | | | Glycol, Glyceryl Poly- | 2.00 |
| | | | Methacrylate | |

Preparation:

The pearlescent pigment was dispersed in the water of phase A and the Carbopol was added with stirring. Following complete dissolution, the predissolved phase B was stirred in. Finally, phases C and D were added.

Comments:

Opaque, gold-lustrous gel

pH (25° C.): 6,5

Viscosity: 60 000 mPa·s (Brookfield RVT, spindle C, 5 rpm, Helipath) at 25° C.

Sources of Supply:

(1) Merck KGaA

(2) BF Goodrich GmbH

(3) ISP Global Technologies

(4) Guardian

### EXAMPLE 16

After-Shave Soft-Cream

| Starting material | Art. No. | INCI-Name | | Wt % |
|---|---|---|---|---|
| A | | | | |
| Eumulgin B1 | | (1) | Ceteareth-12 | 0.50 |
| Eumulgin B2 | | (2) | Ceteareth-20 | 0.50 |
| Cutina MD-V | | (1) | Glyceryl Stearate | 3.00 |
| Cetiol LC | | (1) | Coco-caprylate | 5.00 |
| | | | Caprate | |
| Carbopol Ultrez 10 | | (2) | Carbomer | 0.30 |
| B | | | | |
| Water, demineralized | | | Aqua (Water) | 66.10 |
| Glycerin (87% high-grade) | 104091 | (3) | Glycerin | 3.00 |
| Ethanol (96% high-grade) | 100971 | (3) | Alcohol | 20.00 |
| Menthol, cryst. | 105995 | (3) | Menthol | 0.30 |
| Tris(hydroxymethyl)amino- | 130132 | (3) | Tromethamine | 0.30 |
| Methane | | | | |
| RonaCare ™ Ectoin | 130200 | | Ectoin | 1.00 |

Preparation:

Phases A and B were separately heated to 80° C. Phase A was added to phase B with stirring, and the mixture was homogenized and then cooled to room temperature with stirring.

Comments:

pH (25° C.): 7.25

Viscosity (25° C.): 34 000 mPa·s (Brookfield RVT, spindle C, 5 rpm, Helipath)

Sources of Supply:

(1) Cognis GmbH

(2) BF Goodrich GmbH

(3) Merck KgaA

### EXAMPLE 17

Evening-Creme Containing RonaCare™ Ectoin

| Starting material | Art. No. | INCI-Name | | Wt % |
|---|---|---|---|---|
| A | | | | |
| TIMIRON ® Splendid | 117474 | (1) | CI 77891 (Titanium | 2.00 |
| Gold | | | Dioxide), | |
| | | | Mica, Silica | |
| Carbopol ETD 2001 | | (2) | Carbomer | 0.50 |
| Citronensäure | 102895 | (1) | Citric Acid | q.s. |
| Water, demineralized | | | Aqua (Water) | 40.00 |
| B | | | | |
| Propylene glycol | 107478 | (1) | Propylene Glycol | 3.00 |
| RonaCare ™ Ectoin | 130200 | (1) | Ectoin | 1.00 |
| Water, demineralized | | | Aqua (Water) | 28.45 |
| Preservative | | | | |
| C | | | | |
| Hostaphat KL 340 N | | (3) | Dilaureth-4 Posphate | 3.00 |
| Cetyl alcohol | 100989 | (1) | Cetyl Alcohol | 2.00 |
| Paraffin liquid | 107162 | (1) | Paraffinum Liquidum | 10.00 |
| | | | (Mineral Oil) | |
| Cetiol V | | (4) | Decyl Oleate | 6.00 |
| D | | | | |
| Triethanolamine high-grade | 108377 | (1) | Triethanolamine | 0.35 |
| Water, demineralized | | | Aqua (Water) | 3.50 |
| E | | | | |
| Perfume Vogue 2309334 | | (5) | Parfum | 0.20 |

Preparation:

The pearlescent pigment was dispersed in the water of phase A. The mixture was possibly acidified with some drops of citric acid in order to reduce the viscosity. Carbopol was disseminated with stirring. Following complete dissolution, predissolved phase B was slowly stirred in. Phase A/B and phase C were heated to 80° C., phase C was stirred into phase A/B, and the mixture was homogenized, neutralized with phase D, again homogenized, and cooled with stirring.

Comments:

pH (24° C.): 5.8

Viscosity: 33000 mPa·s (Brookfield RVT, spindle C, 5 rpm, Helipath), 24° C.

### EXAMPLE 18

Pre-Solarium Soft-Creme Containing RonaCare™ Ectoin

| Starting material | Art. No. | INCI-Name | | Wt % |
|---|---|---|---|---|
| A | | | | |
| Paraffin viscous | 107160 | (1) | Paraffinum Liquidum (Mineral | 10.00 |
| | | | Oil) | |
| Cetiol S | | (2) | Dioctylcyclohexane | 2.50 |
| Isopropyl | | (2) | Isopropyl Palmitate | 6.50 |
| palmitate | | | | |
| Miglyol 812 N | | (3) | Caprylic/Capric Triglyceride | 1.00 |
| Sunflower | | (4) | Helianthus annuus (Sunflower | 5.00 |
| seed oil | | | Seed Oil) | |
| OXYNEX ® K | 108324 | (1) | PEG-8, Tocopherol, Ascorbyl | 0.10 |
| liquid | | | Palmitate, Ascorbic Acid, Citric | |
| | | | Acid | |
| B | | | | |
| Carbopol ETD | | (5) | Carbomer | 0.30 |
| 2001 | | | | |
| C | | | | |
| Water, | | | Aqua (Water) | 68.40 |
| demineralized | | | | |
| RonaCare ™ | 130200 | (1) | (Ectoin) | 1.00 |
| Ectoin | | | | |
| Sisterna L70-C | | (6) | Aqua (Water), Sucrose Laurate, | 5.00 |
| | | | Alcohol | |
| Sodium | 105588 | (1) | Sodium Hydroxide | 0.00 |
| hydroxide, | | | | |
| 10% strength | | | | |
| Preservative | | | | 0.00 |
| D | | | | |
| Perfume oil | | (7) | Parfum | 0.20 |
| Nikita | | | | |

Preparation:

Phase B was dispersed in phase A. Predissolved phase C was added to phase A/B with stirring, and the mixture was neutralized and homogenized, and phase D was added with stirring.

Comments:

pH (25° C.): 5.5-6.5

Viscosity: 113000 mPa·s (Brookfield RVT, spindle C, 10 rpm, Helipath), 25° C.

Sources of Supply:

(1) Merck KGaA

(2) Cognis GmbH

(3) Condea Chemie GmbH

(4) Gustav Heess GmbH

(5) BF Goodrich GmbH

(6) Sisterna C.V./Dai-Ichi

(7) Dragoco

### EXAMPLE 19

Rich Night Cream Containing RonaCare™ Ectoin

| Starting material | Art. No. | INCI-Name | | Wt % |
|---|---|---|---|---|
| A | | | | |
| Isolan GI 34 | | (1) | Polyglyceryl 4-Isostearate | 1.00 |
| Abil EM 90 | | (1) | Cetyl Dimethicone Copolyol | 2.00 |
| Paracera W 80 | | (2) | Ceresin (Microcrystalline Wax) | 1.50 |
| Cutina HR | | (3) | Hydrogenated Castor Oil | 0.50 |
| Cetiol V | | (3) | Decyl Oleate | 10.00 |
| Dragoxat EH | | (4) | Octyl Octanoate | 5.00 |
| Miglyol 812 N | | (5) | Caprylic/Capric Triglyceride | 10.00 |
| B | | | | |
| Glycerin (87% | 104091 | (6) | Glycerin | 2.00 |
| high-grade) | | | | |
| Magnesium | 105882 | (6) | Magnesium Sulfate | 1.00 |
| sulfate | | | | |
| heptahydrate | | | | |
| RonaCare ™ | 130200 | (6) | Ectoin | 1.00 |
| Ectoin | | | | |
| Water, | | | Aqua (Water) | 66.00 |
| demineralized | | | | |
| Preservative | | | | |
| C | | | | |
| Perfume oil | | | | q.s. |

Preparation:

Phase A and phase B were separately heated to 80° C. Phase B was added to phase A with stirring. The mixture was homogenized; and phase C was added at ca 35° C. The mixture was cooled to room temperature with stirring.

Comments:

Viscosity: 6500 mPa·s (Brookfield RVT, spindle C, 20 rpm, Helipath), 25° C.

Sources of Supply:

(1) Th. Goldschmidt AG

(2) Paramelt

(3) Cognis GmbH

(4) Dragoco Gerberding & Co. AG

(5) Condea Chemie GmbH

(6) Merck KGaA

### EXAMPLE 20

Skin Care Cream Containing RonaCare™ Ectoin

| Starting material | Art. No. | INCI-Name | | Wt % |
|---|---|---|---|---|
| A | | | | |
| Paraffin viscous | 107160 | (1) | Paraffinum Liquidum (Mineral | 8.00 |
| | | | Oil) | |
| Tego Care 150 | | (2) | Glyceryl Stearate, Steareth-25, | 10.00 |
| | | | Ceteth-20, Stearyl Alcohol | |
| Lanette O | | (3) | Cetearyl Alcohol | 1.50 |
| Isopropyl | | (3) | Isopropyl Palmitate | 5.00 |
| palmitate | | | | |
| Abil Wax 2434 | | (2) | Stearoxy Dimethicone | 1.60 |
| Miglyol 812 N | | (4) | Caprylic/Capric Triglyceride | 2.00 |
| Dow Corning | | (5) | Dimethicone | 0.30 |
| 200 Fluid | | | | |
| (350 cs) | | | | |
| B | | | | |
| Glycerin (87% | 104091 | (1) | Glycerin | 3.00 |
| high-grade) | | | | |
| RonaCare ™ | 130200 | (6) | (Ectoin) | 1.00 |
| Ectoin | | | | |
| water, | | | Aqua (Water) | 67.60 |
| demineralized | | | | |
| Preservative | | | | |
| C | | | | |
| Perfume oil | | | | q.s. |

Preparation:

Phase A was heated to 75° C. Phase B was heated to 80° C. and was added to phase A with stirring, and the mixture was homogenized and phase C added at ca 35° C. The mixture was cooled to room temperature with stirring.

Comments:

pH (25° C.): 5.1

Viscosity: 342000 mPa·s (Brookfield RVT, spindle C, 2.5 rpm, Helipath), at 24° C.

Sources of Supply:

(1) Merck KGaA

(2) Th. Goldschmidt AG

(3) Cognis GmbH

(4) Condea Chemie GmbH

(5) Dow Corning

### EXAMPLE 21

Refreshing Cream Containing RonaCare™ Ectoin (W/O)

| Starting material | Art. No. | INCI-Name | | Wt % |
|---|---|---|---|---|
| A | | | | |
| Paraffin liquid | 107162 | (1) | Paraffinum Liquidum (Mineral | 8.00 |
| | | | Oil) | |
| Arlacel P135 | | (2) | PEG-30 Dipolyhydroxystearate | 5.50 |
| Miglyol 812 N | | (3) | Caprylic/Capric Triglyceride | 4.00 |
| Arlamol HD | | (2) | Isohexadecane | 6.00 |
| B | | | | |
| Water, demineralized | | | Aqua (Water) | 46.00 |
| Glycerin (87% high-grade) | 104091 | (1) | Glycerin | 4.00 |
| RonaCare ™ Ectoin | 130200 | (1) | (Ectoin) | 1.00 |
| Magnesium sulfate heptahydrate | 105882 | (1) | Magnesium Sulfate | 0.50 |
| Preservative | | | | |
| C | | | | |
| Ethanol 96% high-grade | 100971 | (1) | Alcohol | 25.00 |

Preparation:

Phase A and phase B were separately heated to 75° C. Phase B was added to phase A with stirring. The mixture was homogenized, and phase C was added at ca 30° C. The mixture was cooled to room temperature with. stirring.

Comments:

Viscosity: 41000 mPa·s (Brookfield RVT, spindle C, 5 rpm, Helipath), at 24° C.

Sources of Supply:

(1) Merck KGaA

(2) Uniqema

(3) Condea Chemie GmbH

### EXAMPLE 22

Skin Care Cream Containing RonaCare™ Ectoin (W/O)

| Starting material | Art. No. | INCI-Name | | Wt % |
|---|---|---|---|---|
| A | | | | |
| Paraffin viscous | 107160 | (1) | Paraffinum Liquidum (Mineral | 10.00 |
| | | | Oil) | |
| Hostacerin WO | | (2) | Polyglyceryl-2-sesquiiso- | 6.00 |
| | | | stearate, Cera Alba (Beeswax), | |
| | | | Cera Microcristallina (Microcrystalline | |
| | | | Wax), Paraffinum | |
| | | | Liquidum (Mineral Oil), Magnesium | |
| | | | Stearate, Aluminum Stearate | |
| Isopropyl palmitate | | (3) | Isopropyl Palmitate | 8.00 |
| Paracera M | | (4) | Microwax | 3.00 |
| Vaseline | | (5) | Petrolatum | 3.00 |
| B | | | | |
| water, demineralized | | (1) | Aqua (water) | 65.00 |
| Glycerin (87% high-grade) | 104091 | (1) | Glycerin | 4.00 |
| RonaCare ™ Ectoin | 130200 | | (Ectoin) | 1.00 |
| Preservative | | | | |

Preparation:

Phase A and phase B were heated to 80° C. Phase B was added to phase A with stirring. The mixture was homogenized and cooled to room temperature with stirring.

Comments:

Viscosity: 220000 mPa·s (Brookfield RVT, spindle D, 5 rpm, Helipath), at 24° C.

Sources of Supply:

(1) Merck KGaA

(2) Clariant GmbH

(3) Cognis GmbH

(4) Paramelt

(5) Schümann Sabol

### EXAMPLE 23

W/O/W Night Cream Containing RonaCare™ Ectoin

| Starting material | Art. No. | INCI-Name | | Wt % |
|---|---|---|---|---|
| A | | | | |
| Brij 721 P | | (1) | Steareth-21 | 2.00 |
| Brij 72 | | (1) | Steareth-2 | 3.00 |
| Arlacel P135 | | (1) | PEG-30 Dipolyhydroxystearate | 1.50 |
| Jojoba oil | | (2) | Buxus chinensis (Jojoba Oil) | 8.00 |
| RonaCare ™ | 130180 | (3) | Tocopheryl Acetate | 1.00 |
| Tocopheryl | | | | |
| acetate | | | | |
| Lanette O | | (4) | Cetearyl Alcohol | 1.00 |
| Stearic acid | 100671 | (3) | Stearic Acid | 1.50 |
| Mirasil CM 5 | | (5) | Cyclomethicone | 1.00 |
| B | | | | |
| Glycerin (87% | 104091 | (3) | Glycerin | 4.00 |
| high-grade) | | | | |
| Preservative | | | | |
| RonaCare ™ | 130200 | | (Ectoin) | 1.00 |
| Ectoin | | | | |
| Water, | | | Aqua (Water) | 76.00 |
| demineralized | | | | |
| Sodium | 105588 | (3) | Sodium Hydroxide | |
| hydroxide, | | | | |
| 10% ig | | | | |

Preparation:

Phase A and phase B were heated to 75° C. Phase A was slowly stirred into phase B, and the mixture was homogenized, possibly neutralized with sodium hydroxide, and cooled with stirring.

Comments:

pH (22° C.): 5.7

Viscosity 24° C.: 42000 mPa·s (Brookfield RVT, spindle C, 5 rpm, Helipath)

Sources of Supply:

(1) Uniqema

(2) Gustav Heess GmbH

(3) Merck KGaA

(4) Cognis GmbH

(5) Rhodia GmbH

### EXAMPLE 24

Sprayable Sunscreen Lotion Containing RonaCare™ Ectoin

| Starting material | Art. No. | INCI-Name | | Wt % |
|---|---|---|---|---|
| A | | | | |
| EUSOLEX ® 2292 | 105382 | (1) | Octyl Methoxycinnamate, BHT | 7.50 |
| EUSOLEX ® 4360 | 105376 | (1) | Benzophenone-3 | 2.50 |
| EUSOLEX ® HMS | 111412 | (1) | Homosalate | 7.00 |
| Hetester PHA | | (2) | Propylene Glycol Isoceteth-3 | 5.00 |
| | | | Acetate | |
| Volpo S-2 | | (3) | Steareth-2 | 0.40 |
| Volpo S-10 | | (3) | Steareth-10 | 0.80 |
| Pemulen TR-2 | | (4) | Acrylates/C10-30 Alkyl Acrylate | 0.18 |
| | | | Crosspolymer | |
| Performa V 825 | | (5) | Synthetic Wax | 0.80 |
| Dow Corning 200 (100 cs) | | (6) | Dimethicone | 1.00 |
| OXYNEX ® K liquid | 108324 | (1) | PEG-8, Tocopherol, Ascorbyl | 0.10 |
| | | | Palmitate, Ascorbic Acid, Citric Acid | |
| B | | | | |
| RonaCare ™ Ectoin | 130200 | (1) | (Ectoin) | 1.00 |
| Water, demineralized | | | Aqua (Water) | 49.82 |
| Preservative | | | | |
| C | | | | |
| Water, demineralized | | | Aqua (Water) | 20.00 |
| EUSOLEX ® 232 | 105372 | (1) | Phenylbenzimidazole Sulfonic | 1.00 |
| | | | Acid | |
| Propylene glycol | | (7) | Propylene Glycol | 2.00 |
| Triethanolamine high-grade | 108377 | (1) | Triethanolamine | 0.90 |

Preparation:

Phase A and phase B were heated to 80° C. Phase B was added to phase A with stirring, and the mixture was neutralized at room temperature with phase C and homogenized.

Comments:

pH (21° C.): 7.0

Viscosity: watery

Sources of Supply:

(1) Merck KGaA

(2) Paroxite Ltd.

(3) Croda GmbH

(4) BF Goodrich GmbH

(5) New Phase Technologies

(6) Dow Corning

(7) Biesterfeld

### EXAMPLE 25

O/W Cream Containing RonaCare™ Ectoin

| Starting material | Art. No. | INCI-Name | | Wt % |
|---|---|---|---|---|
| A | | | | |
| Tego Care 150 | | (1) | Glyceryl Stearate, | 8.00 |
| | | | Steareth-25, Ceteth-20, | |
| | | | Stearyl Alcohol | |
| Lanette 18 | | (2) | Stearyl Alcohol | 1.00 |
| Isopropyl palmitate | | (2) | Isopropyl Palmitate | 3.00 |
| Jojoba oil | | (3) | Buxus chinensis | 7.00 |
| | | | (Jojoba Oil) | |
| B | | | | |
| RonaCare ™ | 130200 | (4) | (Ectoin) | 1.00 |
| Glycerin (87% | 104091 | (4) | Glycerin | 3.00 |
| high-grade) | | | | |
| Preservative | | | | |
| Water, | | | Aqua (Water) | 77.00 |
| demineralized | | | | |

Preparation:

Phases A and B were heated to 80° C. Phase B was added to phase A with stirring. The mixture was homogenized and stirred until cold.

Comments:

pH (23° C.): 5.4

Viscosity (24° C.): 95000 mPa·s (Brookfield RVT, spindle C, 5 rpm, Helipath)

Sources of Supply:

(1) Goldschmidt AG

(2) Cognis GmbH

(3) Gustav Heess GmbH

(4) Merck KGaA

### EXAMPLE 26

O/W Moisture Cream Containing RonaCare™ Ectoin

| Starting material | Art. No. | | INCI-Name | Wt % |
|---|---|---|---|---|
| A | | | | |
| RonaCare™ Ectoin | 130200 | (1) | (Ectoin) | 1.00 |
| Glycerin (87% high-grade) | 104091 | (1) | Glycerin | 3.00 |
| Preservative | | | | |
| Water, demineralized | | | Aqua (Water) | 76.20 |
| B | | | | |
| Sisterna SP30-C | | (2) | Sucrose Distearate | 2.70 |
| Sisterna SP70-C | | (2) | Sucrose Stearate | 0.90 |
| Cetiol OE | | (3) | Dicaprylyl Ether | 5.00 |
| Miglyol 812 | 106175 | (1) | Caprylic/capric | 2.00 |
| | | | Triglyceride | |
| Isopropyl palmitate | | (3) | Isopropyl Palmitate | 2.00 |
| Cegesoft C 24 | | (3) | Octyl Palmitate | 7.00 |
| Carbopol ETD 2001 | | (4) | Carbomer | 0.20 |
| C | | | | |
| Sodium hydroxide, 10% ig | 105588 | (1) | Sodium Hydroxide | |

Preparation:

Phase A was heated to 75° C., and phase B was well mixed in the cold and then heated to 75° C. Phase B was then added to phase A with stirring, and the mixture was homogenized, neutralized, and stirred until cold.

Comments:

pH (22° C.): 6.5

Viscosity (21° C.): 109000 mPa·s (Brookfield RVT, spindle C, 5 rpm, Helipath)

Sources of Supply:

(1) Merck KGaA

(2) Sisterna C.V./Dai-Ichi

(3) Cognis GmbH

(4) BF Goodrich GmbH

1. A method of using at least one compound, selected from the group comprising compounds of formulas 1a and 1b 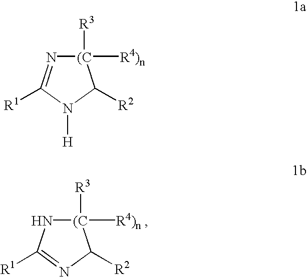
physiologically acceptable salts thereof, and stereoisomeric forms thereof, in which
R¹ denotes H oder alkyl,
R² denotes H, COOH, COO-alkyl or CO—NH—R⁵,
R³ and R⁴ each independently denote H or OH,
n is 1, 2 or 3,
R⁵ denotes H, alkyl, an amino acid group, a dipeptide residue or a tripeptide residue, and
alkyl denotes an alkyl group containing from 1 to 4 carbons for the prophylaxis and/or treatment of UV-induced immunosuppression.

2. A method as defined in claim 1 for the protection of Langerhans cells in the skin

3. A method as defined in claim 1, wherein said compound(s) are present in a topical composition.

4. A method as defined in any one of claims 1 to 3, wherein at least one of the compounds defined in claim 1 is present in a topical composition in a concentration of from 0.0001 to 50 wt %, based on the composition.

5. A method as defined in any one of claims 1 to 4, wherein (S)-1,4,5,6-tetra-hydro-2-methyl-4-pyrimidinecarboxylic acid and/or (S,S)-1,4,5,6-tetrahydro-5-hydroxy-2-methyl-4-pyrimidinecarboxylic acid are used.
